(19) United States
(12) Patent Application Publication
(10) Pub. No.: US 2026/0096849 A1
(43) Pub. Date: Apr. 9, 2026

(54) CONTOURED ELECTRODES FOR PULSED ELECTRIC FIELD ABLATION, AND SYSTEMS, DEVICES, AND METHODS THEREOF

(71) Applicant: Boston Scientific Scimed, Inc., Maple Grove, MN (US)

(72) Inventor: Raju Viswanathan, Palo Alto, CA (US)

(21) Appl. No.: 19/416,879

(22) Filed: Dec. 11, 2025

Related U.S. Application Data
(63) Continuation of application No. 17/469,300, filed on Sep. 8, 2021, now Pat. No. 12,496,123.
(60) Provisional application No. 63/075,729, filed on Sep. 8, 2020.

(51) Int. Cl. A61B 18/14; A61B 18/00; A61B 18/12

(52) U.S. Cl. CPC A61B 18/1492; A61B 2018/0022; A61B 2018/00577; A61B 2018/00613; A61B 2018/126; A61B 2018/1405

(57) ABSTRACT

Systems, devices, and methods are provided herein for delivering pulsed electric fields to ablation tissue (e.g., cardiac tissue). An ablation device can include one or more contoured electrodes with contoured edges that are disposed on an insulating member of the ablation device. The contoured electrodes can be configured to reduce an electric field intensity at an interface between the contoured electrodes and the insulating member and/or a drop-off in electric field intensity.

### CROSS-REFERENCE TO RELATED APPLICATIONS

This application claims priority to U.S. patent application Ser. No. 17/469,300, filed on Sep. 8, 2021, which claims priority to U.S. Provisional Ser. No. 63/075,729 , filed on Sep. 8, 2020, the entire disclosure of which is incorporated herein by reference in its entirety.

### TECHNICAL FIELD

The embodiments described herein relate generally to medical devices for therapeutic electrical energy delivery, and more particularly to systems, apparatuses, and methods for tissue ablation devices (e.g., ablation catheters) having contoured electrodes and generating pulse electric fields using such devices.

### BACKGROUND

Pulsed field ablation using application of high voltage pulses has been demonstrated to be suitable for the rapid and effective ablation of cardiac tissue as well as other target anatomy. In the cardiac context, pulsed field ablation may generate locally high electric fields configured to drive irreversible electroporation (e.g., breakdown cell membranes resulting in cell death). For example, an ablation catheter configured for focal ablation can be used to delivered pulsed field ablation via irreversible electroporation to cardiac tissue. However, high voltage pulses within a fluid medium (e.g., blood) may result in electrolysis and/or associated generation of bubbles. For example, an electric field near an edge of an electrode may be large enough to drive electrical breakdown in gas bubbles and generate local flash arcing. Associated high current densities at an electrode edge can also result in relatively larger bubble sizes. Bubbles and flash arcing are undesirable in the context of clinical applications including cardiac tissue ablation.

### SUMMARY

Described herein are systems, devices, and methods for ablating tissue through irreversible electroporation. In some embodiments, an apparatus includes a first shaft defining a longitudinal axis and a lumen; a second shaft disposed within the lumen and having a distal portion that extends from a distal end of the first shaft; a plurality of electrodes configured to generate an electric field for ablating tissue; and a set of splines, each spline of the set of splines including a set of electrodes of the plurality of electrodes formed on that spline, each set of electrodes including a contoured electrode, each contoured electrode including: a proximal edge and a distal edge, at least one of the proximal edge or the distal edge being a contoured edge, the contoured edge having at least one concave portion or convex portion, the set of splines configured to transition into an expanded configuration in which the set of splines bow radially outward from the longitudinal axis of the first shaft.

In some embodiments, an apparatus includes a linear shaft defining a longitudinal axis; and a plurality of electrodes disposed on a distal portion of the linear shaft, the plurality of electrodes configured to generate an electric field for ablating tissue, the plurality of electrodes including: a tip electrode disposed at a distal end of the linear shaft, the tip electrode including a first contoured edge; and a set of proximal electrodes disposed proximal of the tip electrode, the set of proximal electrodes including a contoured electrode having a second contoured edge, the first and second contoured edges each having at least one concave portion or convex portion.

In some embodiments, a method includes generating a pulse waveform using a signal generator that is coupled to an ablation device, the ablation device including a plurality of electrodes disposed on a distal portion of the ablation device, the distal portion of the ablation device being positioned within a heart of a patient; and delivering the pulse waveform to a set of electrodes of the plurality of electrodes such that subsets of the set of electrodes are energized with opposite polarities to generate a pulsed electric field to ablate tissue near the distal portion of the ablation device, the set of electrodes including at least one contoured electrode disposed on a flexible member of the ablation device, each contoured electrode having a contoured edge that reduces (1) an intensity of the electric field at an interface between the contoured edge and the flexible member and (2) a drop-off in the intensity in a direction extending away from the contoured edge.

### BRIEF DESCRIPTION OF THE DRAWINGS

FIG. 1 is a block diagram of a system for ablation, according to embodiments.

FIG. 2 is a flow diagram of a method for tissue ablation, according to embodiments.

FIG. 3 is a schematic illustration of a contoured electrode, according to embodiments.

FIG. 4 is a perspective view of a contoured electrode, according to embodiments.

FIG. 5A is a side view of a contoured electrode, according to embodiments.

FIG. 5B is a side view of an unrolled contoured electrode, according to embodiments.

FIG. 6 is a side view of an unrolled contoured electrode, according to embodiments.

FIGS. 7A and 7B are side views of unrolled contoured electrodes, according to embodiments.

FIG. 8 is a side view of an unrolled contoured electrode, according to embodiments.

FIGS. 9A-9E are views of unrolled electrodes with two contoured edges, according to embodiments.

FIG. 10 schematically depicts a distal end of an ablation device having a linear shaft with contoured electrodes, according to embodiments.

FIG. 11 schematically depicts a distal end of an ablation device having an expandable structure with contoured electrodes, according to embodiments.

FIG. 12 is a side view of an unrolled contoured electrode of the ablation device depicted in FIG. 11, according to embodiments.

FIG. 13 schematically depicts a distal end of an ablation device having a basket shape and including contoured electrodes, according to embodiments.

FIG. 14 schematically depicts a contoured electrode of the ablation device depicted in FIG. 13, according to embodiments.

FIGS. 15A and 15B are schematic side views of splines or shafts of an ablation device without and with a contoured electrode, respectively, according to embodiments.

FIG. 16 is a graph of electrical field intensities along a side of the shafts as depicted in FIGS. 15A and 15B, according to embodiments.

FIGS. 17A and 17B schematically depict splines or shafts of an ablation device having two adjacent electrodes with contoured edges, according to embodiments.

FIG. 18 schematically depicts shafts or splines or an ablation device having electrodes without and with contoured edges facing one another, and a graph of electric field intensities along a side of the shafts, according to embodiments.

### DETAILED DESCRIPTION

Described herein are systems, devices, and methods for delivering pulsed electric fields to ablate tissue by irreversible electroporation. In some embodiments, the systems, devices, and methods described herein may be used to generate pulsed electric fields with improved (e.g., reduced) local fields and current densities at electrode edges, e.g., by suitable contouring of electrode edges as described herein. Systems, devices, and methods disclosed herein can generate a local electric field in a tissue region sufficient to drive irreversible electroporation while maintaining electric field values below a safe level in a predetermined tissue region.

The term “electroporation” as used herein refers to the application of an electric field to a cell membrane to change the permeability of the cell membrane to the extracellular environment. The term “reversible electroporation” as used herein refers to the application of an electric field to a cell membrane to temporarily change the permeability of the cell membrane to the extracellular environment. For example, a cell undergoing reversible electroporation can observe the temporary and/or intermittent formation of one or more pores in its cell membrane that close upon removal of the electric field. The term “irreversible electroporation” as used herein refers to the application of an electric field to a cell membrane to permanently change the permeability of the cell membrane to the extracellular environment. For example, a cell undergoing irreversible electroporation can observe the formation of one or more pores in its cell membrane that persist upon removal of the electric field.

In some embodiments, an electrode is configured to produce an electric field having an improved spatial uniformity. For example, an ablation device may include a set of contoured electrodes. In some embodiments, an ablation device including a catheter shaft may include one or more of the electrodes described herein. In some embodiments, a linear catheter ablation device may include a catheter shaft and a distal cap. The distal cap may include one or more distal cap electrodes corresponding to any of the electrodes described herein. In some embodiments, catheter devices for cardiac ablation can be made with electrodes in the form of cylindrical rings mounted on a catheter shaft made of a polymeric material. In some embodiments, a balloon ablation device or other ablation device having an expandable structure may have a distally located inflatable balloon or expandable structure with contoured electrodes formed on the balloon or expandable structure.

### Systems

Disclosed herein are systems and devices configured for generating ablating tissue.

Generally, a system described here for ablating tissue with high voltage pulse waveforms. The systems, methods and implementations described in the present disclosure apply to synchronous or asynchronous ablation delivery. Furthermore, as described herein, the systems and devices may be deployed endocardially and/or epicardially to treat cardiac arrhythmias.

Disclosed herein are systems and devices configured for tissue ablation via the selective and rapid application of voltage pulse waveforms to aid tissue ablation, resulting in irreversible electroporation. Generally, a system for ablating tissue described here may include a signal generator and an ablation device having one or more electrodes for the selective and rapid application of direct current (DC) voltage to drive electroporation. As described herein, the systems and devices may be deployed epicardially and/or endocardially to treat cardiac arrhythmias. Voltages may be applied to selected paired subsets of the electrodes, with independent subset selections for anode and cathode electrode selections in order to deliver pulsed electric field ablation therapy. In embodiments, the paired electrode subsets can be predetermined. In some embodiments, a pacing signal for cardiac stimulation may be generated and used to deliver the ablation pulse waveform by the signal generator in synchronization with the pacing signal.

Generally, the systems and devices described herein include one or more catheters configured to ablate tissue in a heart (e.g., a left atrial chamber of a heart). The catheter can include an electrode having at least one edge having a contour including convex and concave portions. In some embodiments, one or more electrodes can be embodied as a ring electrode on a catheter shaft. In some embodiments, one or more electrodes can be embodied in other forms, e.g., including the distal cap electrode of a linear catheter ablation device.

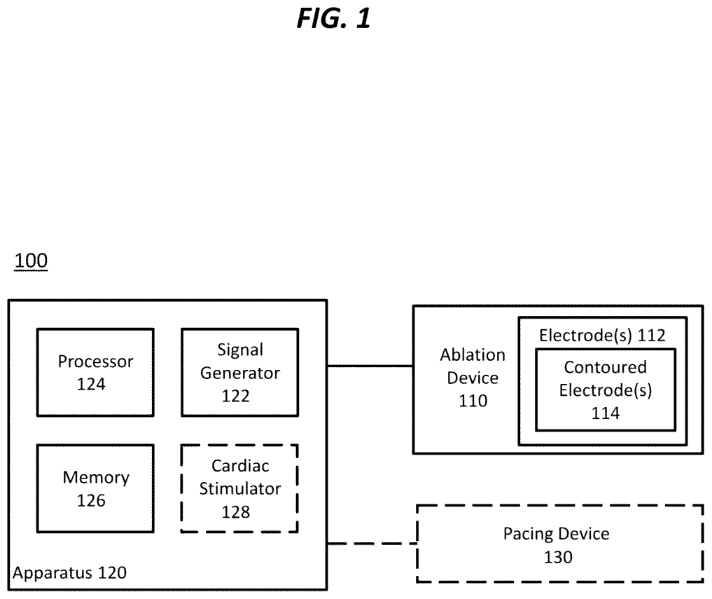

FIG. 1 illustrates an ablation system 100 configured to deliver voltage pulse waveforms. The system 100 may include an apparatus 120 including a signal generator 122, a processor 124, and a memory 126, and optionally a cardiac stimulator 128. The apparatus 120 may be coupled to an ablation device 110 and optionally to a pacing device 130.

The signal generator 122 may be configured to generate pulse waveforms for irreversible electroporation of tissue, such as, for example, pulmonary vein ostia. For example, the signal generator 122 may be a voltage pulse waveform generator and deliver a pulse waveform to the ablation device 110. The processor 124 may incorporate data received from memory 126 to determine the parameters (e.g., amplitude, width, duty cycle, etc.) of the pulse waveform to be generated by the signal generator 122. The memory 126 may further store instructions to cause the signal generator 122 to execute modules, processes and/or functions associated with the system 100, such as pulse waveform generation and/or cardiac pacing synchronization. For example, the memory 126 may be configured to store pulse waveform and/or heart pacing data for pulse waveform generation and/or cardiac pacing, respectively.

In some embodiments, the ablation device 110 can be configured to receive and/or deliver the pulse waveforms described in more detail below. For example, the ablation device 110 may be introduced into an endocardial space of a chamber of the heart (e.g., the left atrium) and placed to position one or more electrodes 112 at one or more pulmonary vein ostia, and then deliver the pulse waveforms to ablate tissue. The ablation device 110 may include one or more electrodes 112, which may, in some embodiments, contain at least one set of independently addressable electrodes. Each electrode may include an insulated electrical lead configured to sustain a voltage potential of at least about 700 V without dielectric breakdown of its corresponding insulation. In some embodiments, the insulation on each of the electrical leads may sustain an electrical potential difference of between about 200 V to about 4,000 V across its thickness without dielectric breakdown. For example, the electrodes 112 may be grouped into one or more paired subsets or anode-cathode subsets (e.g., subsets with electrodes configured to have opposite polarities) such as, for example, a subset including one anode and one cathode, a subset including two anodes and two cathodes, a subset including two anodes and one cathode, a subset including one anode and two cathodes, a subset including three anodes and one cathode, a subset including three anodes and two cathodes, and/or the like.

In some embodiments, the ablation device 110 includes a catheter defining a longitudinal axis. The catheter can include a catheter distal end, a distal portion disposed distally of the catheter distal end, and at least one spline. The spline can include a spline proximal end and a spline distal end, wherein the spline proximal end is coupled to the catheter distal end, and the spline distal end is coupled to the distal portion. Further, the spline can include a first electrode positioned over a portion of a surface of a spline, the first electrode comprising a surface having a proximal boundary and a distal boundary. The proximal boundary and the distal boundary can include closed curves, with the distal boundary located closer to the spline distal end and the proximal boundary located closer to the spline proximal end. Further, the spline can include a second electrode located closer to the spline proximal end than the first electrode.

In some embodiments, the electrodes 112 may include one or more contoured electrodes 114. Contoured electrodes 114 contain one or more contoured edges. Contoured edges can include electrode edges that contain at least one peak or at least one valley, as further described below.

The pacing device 130 may be suitably coupled to the patient (not shown) and configured to receive a heart pacing signal generated by the cardiac stimulator 128 of the apparatus 120 for cardiac stimulation. An indication of the pacing signal may be transmitted by the cardiac stimulator 128 to the signal generator 122. Based on the pacing signal, an ablation voltage pulse waveform may be selected, computed, and/or otherwise identified by the processor 124 and generated by the signal generator 122. In some embodiments, the signal generator 122 is configured to generate the pulse waveform in synchronization with the indication of the pacing signal (e.g., within a common refractory window). For example, in some embodiments, the common refractory window may start substantially immediately following a ventricular pacing signal (or after a very small delay) and last for a duration of approximately 250 milliseconds or less thereafter. In such embodiments, an entire pulse waveform may be delivered within this duration. In alternate embodiments, the ablation pulse waveform can be delivered without a pacing signal, i.e., asynchronously delivered, and therefore a pacing device may not be not needed.

The processor 124 may be any suitable processing device configured to run and/or execute a set of instructions or code. The processor may be, for example, a general-purpose processor, a Field Programmable Gate Array (FPGA), an Application Specific Integrated Circuit (ASIC), a Digital Signal Processor (DSP), and/or the like. The processor may be configured to run and/or execute application processes and/or other modules, processes and/or functions associated with the system and/or a network associated therewith (not shown). The underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

The memory 126 may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, etc. The memory 126 may store instructions to cause the processor 124 to execute modules, processes and/or functions associated with the system 100, such as pulse waveform generation and/or cardiac pacing.

The system 100 may be in communication with other devices (not shown) via, for example, one or more networks, each of which may be any type of network. A wireless network may refer to any type of digital network that is not connected by cables of any kind. However, a wireless network may connect to a wireline network in order to interface with the Internet, other carrier voice and data networks, business networks, and personal networks. A wireline network is typically carried over copper twisted pair, coaxial cable or fiber optic cables. There are many different types of wireline networks including, wide area networks (WAN), metropolitan area networks (MAN), local area networks (LAN), campus area networks (CAN), global area networks (GAN), like the Internet, and virtual private networks (VPN). Hereinafter, network refers to any combination of combined wireless, wireline, public and private data networks that are typically interconnected through the Internet, to provide a unified networking and information access solution.

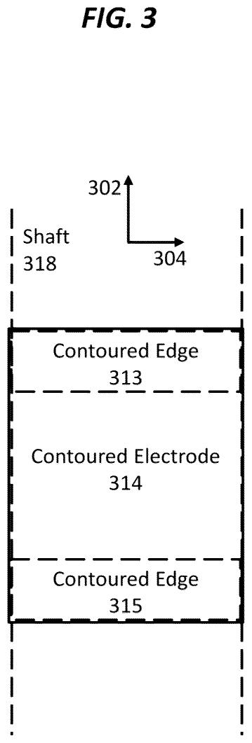

FIG. 3 is a schematic illustration of a contoured electrode 314 with contoured edges 313 and 315 disposed on a shaft or spline 318, according to various embodiments. The shaft or spline 318 may belong to an ablation device such as, for example, the ablation device 110, as described above. The ablation device can include one or more splines or shafts 318 with electrodes (including contoured electrode 314) disposed at discrete locations along a length of the splines or shafts. In some cases, the shaft 318 can include a cross-section that is substantially constant. For example, the shaft 318 can have a cross-section that is substantially circular, elliptical, etc. Alternatively, the shaft 318 can includes sections that have differing cross-sections.

Each contoured electrode 314 can have first and second edges, with at least one contoured edge 313, 315. For example, a contoured electrode 314 can have a single contoured edge 313. Alternatively, a contoured electrode 314 can have two contoured edges 313, 315. Contoured edges 313, 315 can be edges that include at least one peak and at least one valley when viewed along a width of the shaft 318. For example, shaft 314 can have a longitudinal axis 302 and a lateral axis 304 (i.e., an axis extending along a width of the shaft 314). A straight or non-contoured edge can be an edge that extends parallel to the axis 304, while a contoured edge (e.g., edge 313 or 315) can be an edge that has at least one portion that is angled (or has a unit tangent that is angled) relative to axis 304. In some embodiments, a contoured edge can have a periodic curve, with multiple peaks and valleys, while in other embodiments, a contoured edge can have a single peak and a single valley.

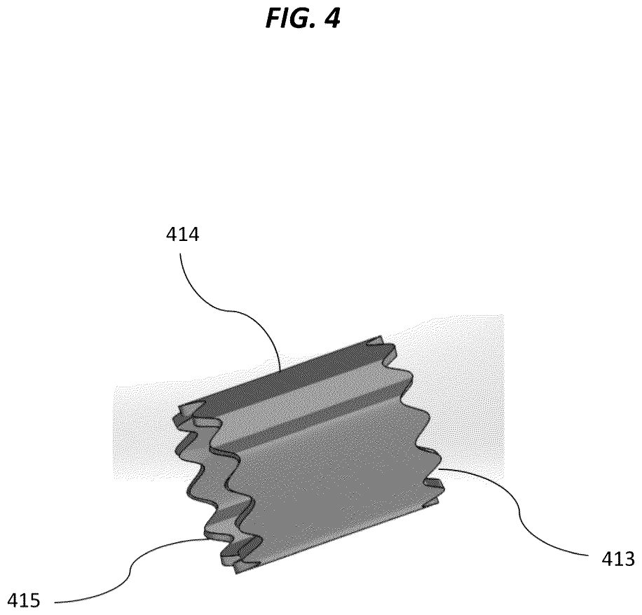

FIG. 4 is a perspective view of an electrode 414 comprising a first edge 413 and a second edge 415, according to embodiments. The electrode 414 can be an example of a contoured electrode 114, 314, as described above with respect to FIGS. 1 and 3, respectively. For example, the electrode 414 can be disposed on a spline or shaft (e.g., shaft 318) of an ablation device (e.g., ablation device 110). The first edge 413 may be opposite the second edge 415. For example, the first edge 413 may be a proximal edge while the second edge 415 may be a distal edge, or vice-versa. The electrode 414 may be, for example, generally cylindrical or annular (e.g., ring-like) in shape. In some embodiments, the electrode 414 may include a circular, ellipsoidal, flattened ellipse, or oval cross-section.

In some embodiments, the edges 413, 415 may include a non-linear shape. For example, the edges 413, 415 may include a generally wave-like pattern. As shown in FIG. 4, the edges 413, 415 may be contoured edges that are shaped (e.g., contoured) in a wave shape having peaks and valleys. In some embodiments, an edge 413, 415 may include one or more patterns. In some embodiments, an edge pattern may be one or more of sinusoidal or curvilinear. In some variations, an edge pattern may be periodic. In some embodiments, the edges 413, 415 may be rounded (e.g., smooth) or may include corners.

In some embodiments, the edges 413, 415 may be symmetric or not symmetric relative to itself or the other edge. In some embodiments, the edges may be identical or different relative to the other edge. In some embodiments, the edges may extend along a circumference of the electrode. In some embodiments, the contour may extend along a portion of the circumference of the electrode.

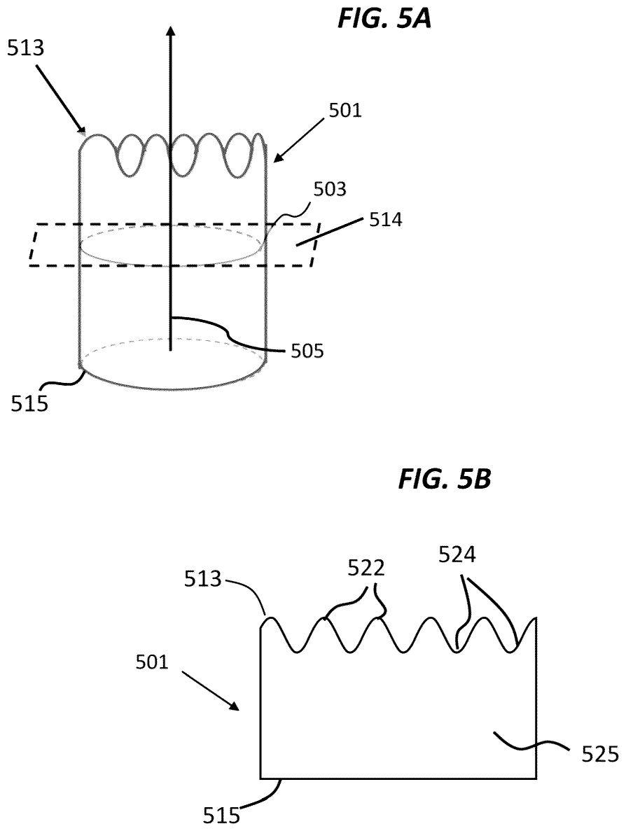

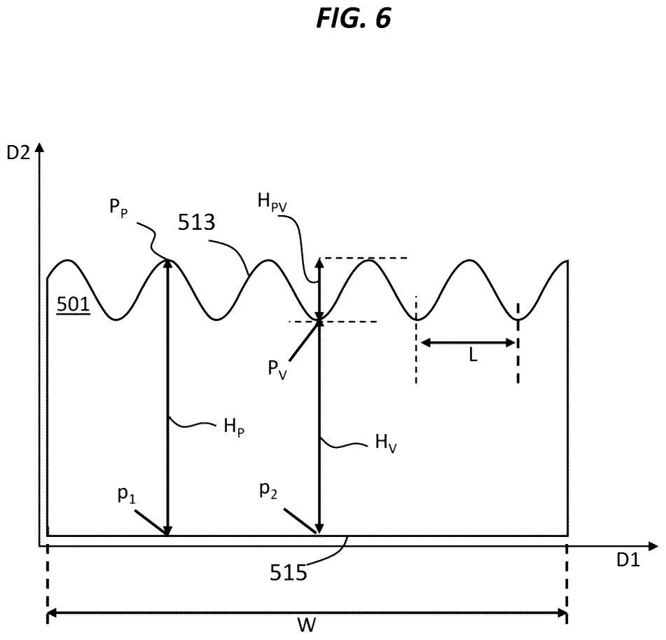

FIG. 5A shows an example of an electrode 501, according to embodiments. The electrode 601 can be an example of a contoured electrode 114, 314, as described above with respect to FIGS. 1 and 3, respectively. For example, the electrode 501 can be disposed on a spline or shaft (e.g., shaft 318) of an ablation device (e.g., ablation device 110). Electrode 501 may include a first edge 513 and a second edge 515. The first edge 513 may be a proximal edge while the second edge 515 may be a distal edge, or vice-versa. In an example embodiment, a cross-section 503 may be a constant shape (constant cross-section along the longitudinal axis). Distal edge 513 may be contoured (e.g., wavy) while proximal edge 515 may not be contoured (herein, such edge is referred to as a non-contoured edge, a straight edge, a flat edge, or an in-plane edge). Non-contoured edge 515 may lie in a single plane that is perpendicular to a longitudinal axis 505 of the electrode 501.

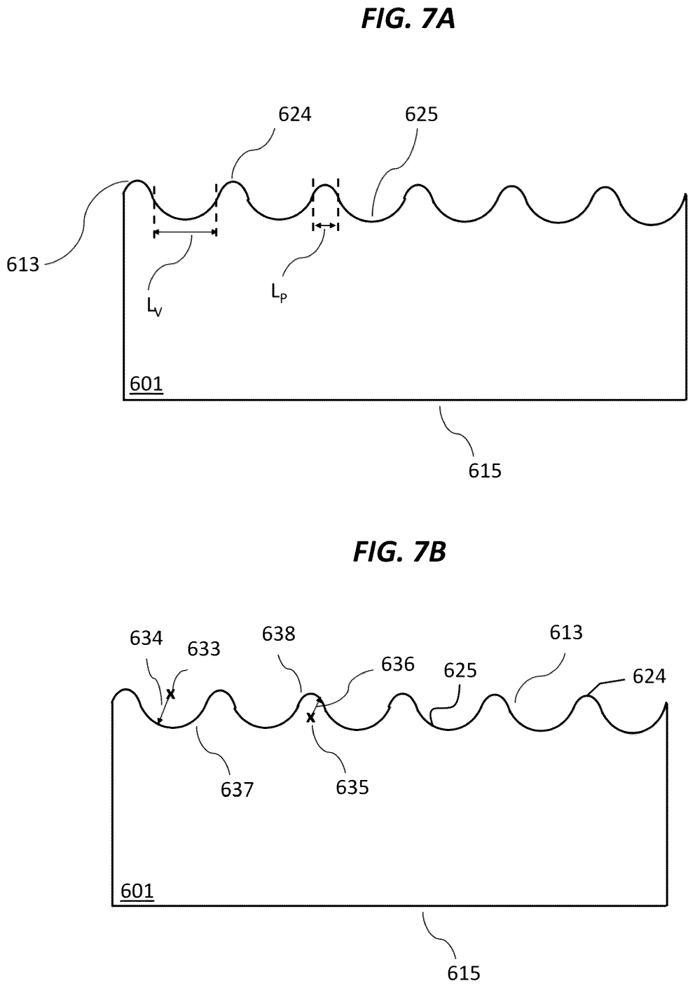

FIGS. 5B and 6 show views of the electrode 501 cut in a direction parallel to axis 505 and unrolled (e.g. flattened). In this rendering, a proximal edge 515 is a straight line, and distal edge 513 includes a set of peaks 522 and valleys 524. A surface of electrode 501 is represented by region 525. In various embodiments, region 525 may be made from any suitable electrically conductive material (e.g., a metal) that is biocompatible. As shown in FIG. 5A, a selected transverse plane 514 intersects electrode 501 and is perpendicular to electrode or spline axis 505. The cross-section 503 lies within transverse plane 514. In some embodiments, the electrode 501 may include a circular, ellipsoidal, flattened ellipse, or oval cross-section 503.

In various embodiments, peaks 522 can be convex portions, and the valleys 524 can be concave portions. For example, one or more convex portions (e.g., peak portions 522) of distal edge 513 may be configured to bulge outward such that points on a line connecting any two points on that convex portion lie inside the area bounded by the borders of the electrode 501. Similarly, one or more concave portions (e.g., valleys 524) may be configured such that points on a line connecting any two points on that concave portion lie outside the area bounded by the borders of the electrode 501. The convex and concave portions may each include one or more generally curvilinear sections.

As depicted in FIG. 6, electrode 501 may have an associated circumferential direction D1 and an associated longitudinal direction D2. In an example embodiment, a distance H_{P} from a peak point P_{P} of edge 513 and an associated closest point p₁ of edge 515 is larger than a distance H_{V} from a valley point P_{V} of edge 513 and an associated closest point p₂ of edge 515. In an example embodiment, H_{P} may be a largest distance between closest points of edges 513 and 515 and H_{V} may be the smallest distance between closest points of edges 513 and 515. As shown in FIG. 6, H_{P} corresponds to a distance between a peak of edge 513 and an associated closest point of edge 515, while H_{V} corresponds to a distance between a valley of edge 513 and an associated closest point of edge 515.

Contoured edge 513 can have a wave shape characterized by a wavelength and a peak height or wave depth. As shown in FIG. 6, difference between H_{P} and H_{V} corresponds to a depth of a wave and is given by H_{P}−H_{V} =H_{PV}. Additionally, the contoured edge 513 may be characterized by a predetermined wavelength L. In some embodiments, a ratio of H_{PV} (i.e., the wave depth) to H_{P} (i.e., a maximum electrode length along its longitudinal axis) may be between about 0.05 and about 0.75, including all values and sub-ranges in-between.

In various embodiments, a number of peaks for contoured edge 513 may depend on (1) a width W of edge 513, which can correspond to a circumference of a spline or shaft of an ablation device, and (2) the wavelength L. For example, a number of peaks N_{P} is given by N_{P}˜W/L. In embodiments described herein, edge 513 may have about one, about two, about three, about four, about five, about six, about seven, about eight, about nine, about ten, or more peaks with an associated number of valleys.

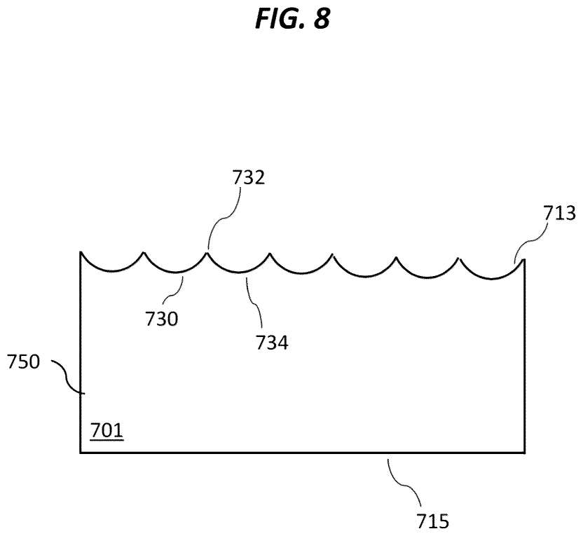

FIGS. 7A and 7B show unrolled (e.g., opened out) views of electrode 601 (e.g., ring electrode) including a first edge 613 and a second edge 615, according to embodiments. The electrode 601 can be an example of a contoured electrode 114, 314, as described above with respect to FIGS. 1 and 3, respectively. For example, the electrode 601 can be disposed on a spline or shaft (e.g., shaft 318) of an ablation device (e.g., ablation device 110). The first edge 613 may be a proximal edge while the second edge 615 may be a distal edge, or vice-versa. The first edge 613 can be a contoured edge and have a wave shape, while the second edge 615 can be a non-contoured edge and be straight in this unrolled rendering. The first edge 613 may include a set of peaks 624 (e.g., convex peaks) and valleys 625 (e.g., concave valleys). In some embodiments, each valley 625 may have the same first length L_{V} and each peak 624 may have the same second length L_{P}. In some embodiments, the first length L_{V} may be different from the second length L_{P}. As shown in FIG. 7A, the first and second lengths are measured in a circumferential direction, as described above with reference to FIG. 6.

Alternatively, in some embodiments, first length L_{V} may be the same as (or smaller than) second length L_{P}. When L_{V}=L_{P}, such embodiment is referred to as a symmetric embodiment. Alternatively, in some embodiments, one or more peaks 624 may have unequal lengths and/or one or more valleys 625 may have unequal lengths, e.g., the valley lengths and peak lengths can generally be different. In such embodiments, the wave can be referred to as an asymmetric wave. In some embodiments, the electrode 601 can have a single peak 624 and a single valley 625, while in alternative embodiments, the electrode 601 can have multiple peaks 624 and/or multiple valleys 625.

In some embodiments, a longest dimension of a transverse cross section of the electrode 601 (e.g., a diameter of the electrode 601) may be between about 0.5 mm and about 6 mm, including all values and sub-ranges in-between. In some embodiments, an electrode length may be between about 0.5 mm and 9 mm, including all values and sub-ranges in-between. In some embodiments, a length of a peak of the first edge 613 may be between about 20 μm and about 20,000 μm, including all values and sub-ranges in-between. In some embodiments, a length of a valley of the first edge 613 may be between about 20 μm and 20,000 μm, including all values and sub-ranges in-between.

Valleys 625 and peaks 624 may be characterized by an associated curvature. In an example embodiment, a valley may have a first center of curvature 633, as shown in FIG. 7B. The first center of curvature 633 may lie outside the area bounded by the borders of the electrode 601 and may include an associated first radius of curvature 634, as shown in FIG. 7B. A second center of curvature 635 can correspond to a peak 625. Second center of curvature 635 may lie inside the area bounded by the borders of the electrode 601 and may include an associated second radius of curvature 636, as shown in FIG. 7B.

In some embodiments, the smallest radius of curvature 634 of a concave portion 637 of a valley 625 may be at least about 10 μm. In some embodiments, the smallest radius of curvature 636 of a convex portion 638 of a peak 624 can be at least about 10 μm. In some embodiments, the largest radius of curvature 634 of one or more concave portions 637 may be less than about 50,000 μm. In some embodiments, the largest radius of curvature 636 of one or more convex portions 638 may be less than about 500 μm. In some embodiments, one or more of the convex portions 638 and concave portion 637 of the edge 613 of the electrode 601 may include or be shaped as a circular arc. In some embodiments, a ratio of the radius of curvature of at least one concave arc to that of at least one convex arc may be greater than about 10.

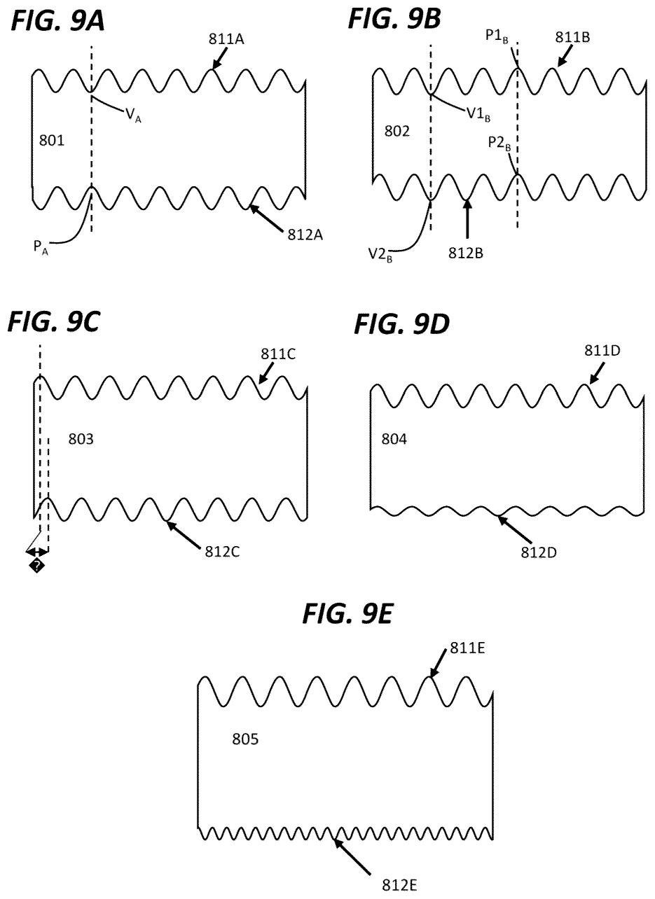

In some embodiments, a point of tangent discontinuity may be included as a peak or a valley of a contoured edge of an electrode. FIG. 8 is a side view in an unrolled rendering of an electrode 701 (e.g., cylindrical ring electrode) including first edge 713 and second edge 715, according to embodiments. The electrode 701 can be an example of a contoured electrode 114, 314, as described above with respect to FIGS. 1 and 3, respectively. For example, the electrode 701 can be disposed on a spline or shaft (e.g., shaft 318) of an ablation device (e.g., ablation device 110). The first edge 713 may be opposite the second edge 715. For example, the first edge 713 may be a proximal edge while the second edge 715 may be a distal edge, or vice-versa. The first edge 713 may include a set of concave portions, such as portions 730, 734. In some embodiments, the first edge 713 may have one or more tangent discontinuities between adjacent concave portions 730 and 734, which may be peaks 732 of electrode 701. That is, peaks 732 can be tangent discontinuity points.

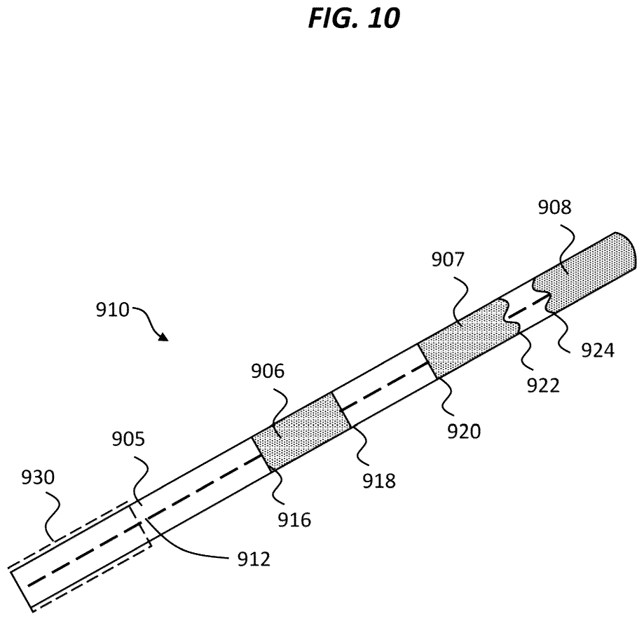

FIGS. 9A-9E show example electrodes 801-805 with contoured first edges 811A-811E and contoured second edges 812A-812E, according to various embodiments. As shown, contoured edges 811A-811E, 812A-812E can be symmetrical and/or different in wavelength and/or wave depth. The electrodes 801-805 are shown unrolled (e.g., opened out) in FIGS. 9A-9E. The electrodes 801-805 can be examples of a contoured electrode 114, 314, as described above with respect to FIGS. 1 and 3, respectively. For example, the electrodes 801-805 can be disposed on a spline or shaft (e.g., shaft 318) of an ablation device (e.g., ablation device 110).

In an example embodiment shown in FIG. 9A, the edges 811A, 811B of electrode 801 can be symmetric with respect to one another in reflection, e.g., edge 812A can have reflectional symmetry with respect to edge 811A such that a peak of edge 812A lines up with a valley of edge 811A. In particular, a peak P_{A} lines up with a valley V_{A}, as shown in FIG. 9A. Alternatively, in an example embodiment shown in FIG. 9B, edges 811B, 812B of electrode 802 can be symmetric with respect to one another in translation, e.g., edge 812B can have translational symmetry to edge 811B such that a valley V1_{B} lines up with a valley V2_{B}, and a peak P1_{B} lines up with a peak P2_{B}. Alternatively, FIG. 9C shows electrode 803 with contoured edges 811C and 812C forming a phase shifted pattern characterized by edge 812C having the same peaks and valleys as edge 811C, but phase shifted by a predetermined value of φ. Alternatively, FIG. 9D shows electrode 804 for which contoured edges 811D and 812D differ in amplitude. For example, edge 812D can have a smaller amplitude of peaks or wave depth relative to edge 811D. Alternatively, FIG. 9E shows an embodiment of electrode 805 with contoured edges 811E and 812E that have different wavelengths and wave depths. For example, edge 812E may have a smaller wavelength and therefore a larger number of peaks and valleys than edge 811E.

While FIGS. 9A and 9E depict different variations of electrodes having two contoured edges, it can be appreciated that any edge configurations may be combined in any suitable way. For instance, an amplitude varying pattern (FIGS. 9D and 9E) may be combined with a phase shifted pattern (FIG. 9C).

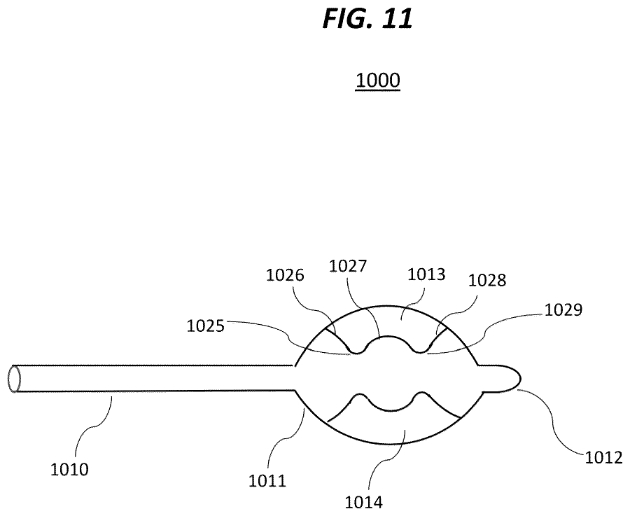

FIG. 10 shows a distal end of an example ablation device 910, according to embodiments. Ablation device 910 can be structurally and/or functionally similar to other ablation devices described herein, including, for example, ablation device 110 described with reference to FIG. 1. Ablation device 910 can be an example of a linear ablation device. For example, ablation device 910 includes a shaft 905 and a plurality of electrodes 906, 907, and 908 disposed along shaft 905. In some embodiments, the plurality of electrodes 906, 907, and 908 may be configured to deliver ablation therapy, including high voltage electrical pulses for pulsed electric field ablation or irreversible electroporation. A sleeve or sheath 930 can optionally be disposed over a portion of the shaft 905. The sheath 930 and the shaft 905 can be configured to move relative to one another to expose a greater number or portion of electrodes disposed along the shaft 905. In such manner, the sleeve 930 can be used to reduce or increase a number of electrodes exposed outside of the sleeve 930 and are available for delivering ablation. Suitable examples of linear ablation devices are described in International Application Serial No. PCT/US2020/037948, filed on Jun. 16, 2020, and titled “SYSTEMS, DEVICES, AND METHODS FOR FOCAL ABLATION,” the contents of which are hereby incorporated by reference in their entirety.

In some embodiments, ablation device 910 may be configured to be steered or deflected by a suitable deflection control mechanism, such as, or example, a pull wire 912. In some embodiments, one or more portions of the shaft 905 may be configured to be flexible and may be bendable via the deflection control mechanism 912. Additionally, or alternatively, one or more portions of the shaft 905 may be rigid, such as portions where electrodes 906, 907, and 908 are disposed.

In some embodiments, a distal end of ablation device 910 may include a distal tip electrode 908, e.g., implemented as a distal cap electrode. Electrodes 906 and 907 can be disposed proximal to distal tip electrode 908. In some embodiments, electrodes 907 and 908 may be generally cylindrical in shape (e.g., ring-shaped) with a proximal edge and a distal edge. For example, electrodes 906 and 907 may have respective edges 916, 918 and 920, 922. Distal tip electrode 908 may have a single edge 924. In some embodiments, one or more of edges 916, 918, 920, 922, 924 may be contoured edges. For example, as depicted in FIG. 10, distal edge 922 of electrode 907 and proximal edge 924 of distal tip electrode 908 may each have a contoured (e.g., wave, curvilinear) shape, as further described herein. In some embodiments, one or more peaks or valleys of contoured edges 922 and 924 may be aligned (e.g., in-line) along a longitudinal axis of ablation device 910. Additionally, or alternatively, one or more peaks or valleys of the contoured edges 922 and 924 may not be aligned with each other.

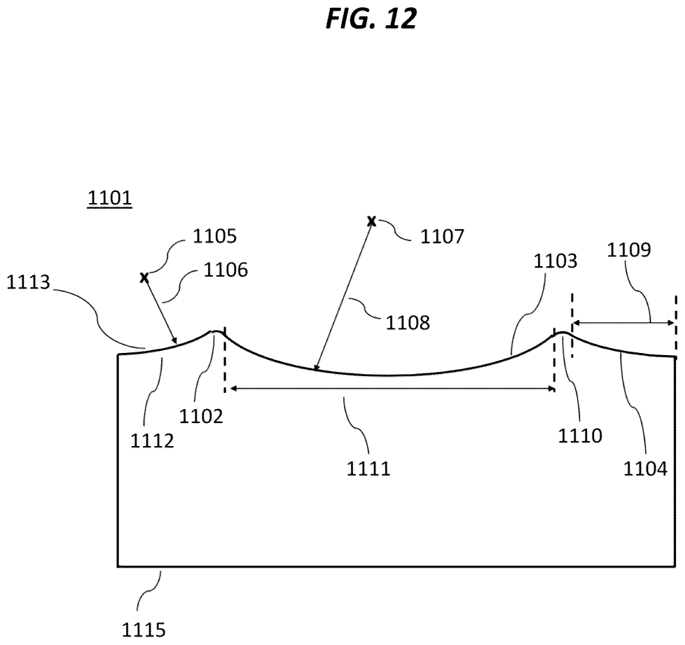

FIG. 11 shows a distal end of an example ablation device 1000, according to embodiments. Ablation device 1000 can be structurally and/or functionally similar to other ablation devices described herein, including, for example, ablation device 110 described with reference to FIG. 1. Ablation device 1000 can be an example of a balloon ablation device or other ablation device with an expandable or inflatable structure. For example, ablation device 1000 can include a catheter shaft 1010 and an inflatable member 1011 (e.g., balloon) disposed at a distal portion of the ablation device 1000. Suitable examples of balloon ablation devices, such as for focal ablation, are described in International Application Serial No. PCT/US18/50660, filed on Sep. 12, 2018, titled “SYSTEMS, APPARATUSES, AND METHODS FOR VENTRICULAR FOCAL ABLATION,” and published as International Application Publication No. WO 2019/055512, the contents of which are hereby incorporated by reference in their entirety.

In some embodiments, the ablation device 1000 may include a distal tip 1012. In some embodiments, the inflatable member 1011 may include one or more electrodes 1013 and 1014 disposed on a surface of the inflatable member 1011. For example, the inflatable member 1011 may be composed of a polymeric material, and the electrodes 1013 and 1014 may be composed of a metallic film deposited on the inflatable member 1011. As depicted in FIG. 11, the electrodes 1013 and 1014 may be contoured as described herein and may include one or more convex portions and concave portions along one or more edges. For example, electrode 1013 includes an edge having concave portions 1026, 1027, and 1028, and convex portions 1025 and 1029.

In some embodiments, the shaft 1010 may define a lumen configured for a guidewire (not shown) to be slidably disposed. For example, a guidewire may be configured for over-the-wire delivery of the ablation device 1000 to a predetermined location within the patient. While not depicted in FIG. 11, in some embodiments, catheter shaft 1010 may also include one or more contoured electrodes, with contours having any of the structures described herein.

In various embodiments, electrodes 1013 and 1014 are configured to be stretchable or expandable. Any suitable approaches may be used for forming stretchable electrodes (e.g., electrodes formed using a network of conductive overlapping filaments, such as metallic nanowires, embedded in (or disposed over) a stretchable and flexible substrate. In some cases, a stretchable electrode may be formed from a network of folded conductive elements which are configured to unfold to allow the stretchable electrode to stretch in one or more directions.

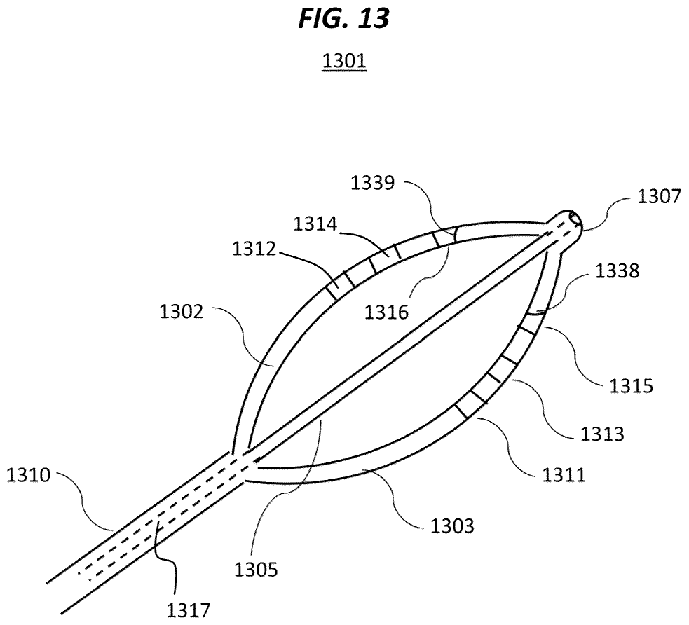

In some embodiments, an electrode, such as the electrodes 1013, 1014 depicted in FIG. 11, may include a plurality of concave portions and/or plurality of convex portions (e.g., a plurality of waves) having unequal lengths and/or radii of curvature. The electrode can be positioned on a balloon ablation catheter, as depicted with respect to FIG. 11, or the electrode can be positioned on a shaft or spline of a linear ablation catheter or basket ablation catheter. For example, FIG. 12 shows an example view of a ring electrode 1101 in an unrolled configuration with a contoured edge 1113 that includes a plurality of concave portions and/or plurality of convex portions (e.g., a plurality of waves) having unequal lengths and/or radii of curvature. The electrode 1101 can be an example of a contoured electrode 114, 314, as described above with respect to FIGS. 1 and 3, respectively. For example, the electrode 1101 can be disposed on a spline or shaft (e.g., shaft 318) of an ablation device (e.g., ablation device 110).

Electrode 1101 includes a first edge 1113 and a second edge 1115. For example, first edge 1113 may have a generally asymmetric and non-uniform wave shape, while second edge 1115 may be straight (i.e., non-contoured). The first edge 1113 may include a set of peaks 1102, 1110 and valleys 1103, 1104, 1112. Peaks 1102, 1110 may be convex portions, and valleys 1103, 1104, 1112 may be concave portions. First edge 1113 may include generally asymmetric waves.

In some embodiments, a first center of curvature 1105 corresponds to valley 1112. The first center of curvature 1105 may lie outside the area bounded by the borders of the electrode 1101 and may include an associated first radius of curvature 1106. A second center of curvature 1107 corresponds to valley 1103. The second center of curvature 1107 may lie outside the area bounded by the borders of the electrode 1101 and may include an associated second radius of curvature 1108. The first radius of curvature 1106 may be different from the second radius of curvature 1108. In some embodiments, valleys may have different lengths. For example, valley 1104 may have a first length 1109 and valley 1103 may have a second length 1111.

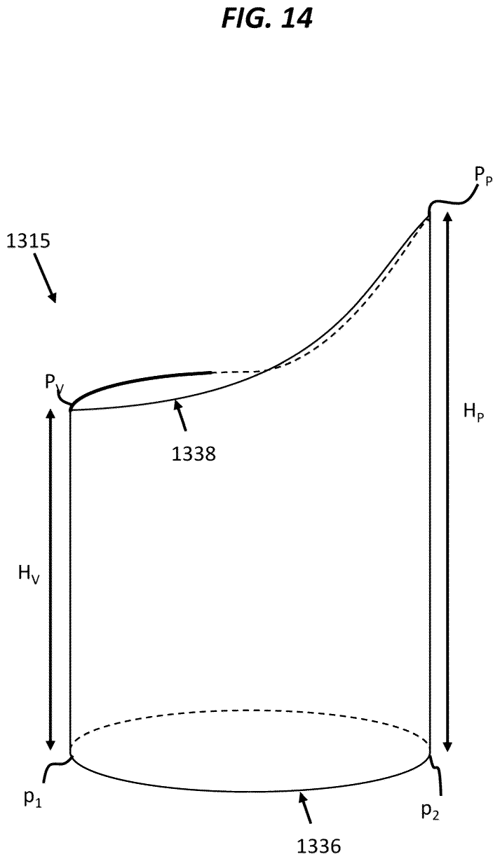

FIG. 13 shows ablation device 1301, having electrodes 1311-1316, according to embodiments. Ablation device 1301 can be structurally and/or functionally similar to other ablation devices described herein, including, for example, ablation device 110 described with reference to FIG. 1. Ablation device 1301 can be an example of a basket ablation device or other type of expandable ablation device.

Ablation device 1301 includes a shaft 1310 at a proximal end of the device 1301, a guidewire lumen 1317, a distal tip 1307, and one or more splines 1302, 1303, as shown in FIG. 13. The guidewire lumen 1317 may be disposed within the shaft 1310 and configured to extend to the distal tip 1307. A guidewire (not shown) may be configured to be slideably disposed within the guidewire lumen 1317. For example, a guidewire may be configured for over-the-wire delivery of the ablation device 1301 to predetermined location within the patient.

In some embodiments, one or more of the splines 1302, 1303 may include one or more electrodes (e.g., ring electrodes) disposed along its length. For example, electrodes 1311, 1313, and 1315 can be disposed on spline 1303, and electrodes 1312, 1314, and 1316 can be disposed on spline 1302. Distal tip 1307 may include an atraumatic shape to reduce trauma to tissue. A proximal end of the set of splines 1302, 1303 (or of a single spline, if only one spline is used) may be coupled to a distal end of the catheter shaft 1310, and a distal end of the set of splines 1302, 1303 (or of a single spline, if only one spline is used) may be coupled to the distal tip 1307 of device 1301.

Ablation device 1301 may be configured for delivering a pulse waveform to tissue via the electrodes 1311-1316 disposed on the splines 1302, 1303. In an example embodiment, any suitable number of splines may be used (e.g., there can be one, two, three, four, five, six, seven, eight, nine, ten, etc. splines). In example embodiments, ablation device 1301 includes 3-20 or more splines, including all values and sub-ranges in between. In some cases, ablation device 1301 may include more than 20 splines.

Splines 1302 and 1303 may include one or more jointly wired or independently addressable electrodes 1311-1316 formed on surfaces of splines 1302, 1303. Each electrode (e.g., electrodes 1311-1316) may include an insulated electrical lead configured to sustain a voltage potential of at least about 700 V without dielectric breakdown of its corresponding insulation. In some embodiments, the insulation on each of the electrical leads may sustain an electrical potential difference of between about 200 V and about 4,000 V across its thickness without dielectric breakdown. Each spline 1302, 1303 can include or contain the insulated electrical leads of those electrodes that are formed on a body of that spline 1302, 1303 (e.g., within a lumen of the spline). In cases where the electrodes on a single spline are wired together, a single insulated lead may carry strands connecting to different electrodes on the spline.

In some embodiments, distal electrodes 1315, 1316 (and/or any of the electrodes) may include distal edges 1338, 1339, respectively, having a contoured shape as described herein. In some embodiments, different splines can include electrodes having different contouring and/or distal and proximal electrodes on each spline can include electrodes having different contouring, according to embodiments described herein. The electrodes on each spline 1302, 1303 can be grouped in a distal electrode (i.e., electrodes 1315, 1316) and a set of proximal electrodes (i.e., electrodes 1311, 1312, 1313, 1314). In some embodiments, proximal electrodes 1311, 1312, 1313, 1314 can be substantially similar in size and shape. Proximal electrodes 1311 and 1313 (and similarly proximal electrodes 1312 and 1314) can be spaced from one another by a first distance, and a distal edge of proximal electrode 1313 and a proximal edge of distal electrode 1315 (and similarly a distal edge of proximal electrode 1314 and a proximal edge of distal electrode 1316) can be spaced from one another by a second distance. In some embodiments, the first and second distances can be different. For example, the second distance can be greater than the first distance. Alternatively, the first and second distances can be the same. While certain spacing and arrangements of electrodes are described herein, it can be appreciated that the size, shape, and spacing of the electrodes may differ without departing from the scope of the present disclosure.

In various embodiments, splines 1302 and 1303 may be flexible. In some embodiments, the ablation device 1301 may be configured to be deployed by pulling on the guidewire lumen 1317 by an actuation mechanism disposed at a handle (not shown) of the ablation device 1301. As the guidewire lumen 1317 is pulled in along the shaft 1310, the splines 1302, 1303 may be configured to bow outward in an expanded configuration (e.g., in a basket-like shape). In some embodiments, the ablation device 1301 may not have a guidewire lumen and may instead include a deflection mechanism (e.g., a pull wire) for steering the ablation device 1301 towards a predetermined anatomical target. In some cases, the splines 1302, 1303 can be configured to transform between a first configuration (e.g., undeployed configuration) where the splines 1302, 1303 are arranged substantially parallel to the longitudinal axis of the ablation device 1301 and a second configuration (e.g., expanded or deployed configuration) where the splines 130, 1303 bow radially outward from the longitudinal axis of the ablation device 1301 (or a longitudinal axis of the shaft 1310). In some embodiments, the splines 1302, 1303 in the second configuration can form a basket having an asymmetric shape along its length, so that one end (for example, the distal end) of the basket is more bulbous than the other end (for example, the proximal end) of the basket.

In some embodiments, when suitably deployed, the splines may be configured to form a nearly planar petal-like configuration (e.g., flower configuration) or to form a basket. For example, ablation devices disclosed herein may include structural and/or functional components similar to those described in U.S. Pat. No. 10,172,673, filed on Sep. 21, 2017, and titled “SYSTEMS DEVICES, AND METHODS FOR DELIVERY OF PULSED ELECTRIC FIELD ABLATIVE ENERGY TO ENDOCARDIAL TISSUE,” and U.S. Pat. No. 10,660,702, filed on Apr. 3, 2020, and titled “SYSTEMS, DEVICES, AND METHODS FOR FOCAL ABLATION,” the contents of each of which are hereby incorporated by reference in their entirety.

In one embodiment, proximal electrodes 1311, 1312, 1313, 1314 can be configured to have a first polarity while distal electrodes 1315, 1316 can be configured to have a second polarity opposite the first polarity. In such matter, subsets of proximal electrodes 1311, 1312, 1313, 1314 and distal electrodes 1315, 1316 can be configured to act as paired anode-cathode subsets. Alternatively, different combinations of electrodes can be selected to act as anode-cathode subsets. The paired anode-cathode subsets can be configured to deliver pulsed field ablation to tissue, as described with reference to FIG. 1 above.

Referring more specifically to the contouring of distal electrodes 1315, 1316, these distal electrodes can have contoured edges 1338, 1339, respectively, that are selected to reduce an electrical field intensity below a target value in the vicinity of the contoured edges 1338, 1339. Such reduction in the electrical field intensity may alleviate electrolysis and/or associated generation of bubbles within a medium in which the ablation device 1301 is placed (e.g., within a liquid medium such as blood).

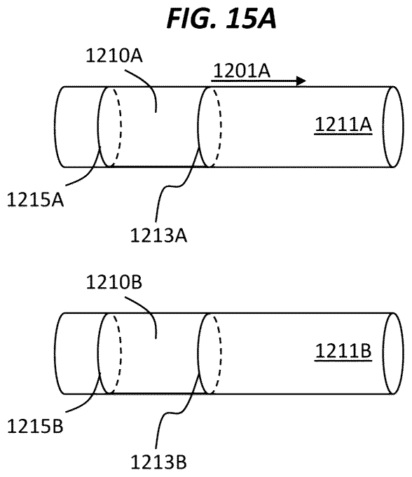

FIG. 14 is an enlarged view of electrode 1315 of ablation device 1301. As shown, electrode 1315 includes a first edge 1338 and a second edge 1336. The first edge 1338 may be opposite the second edge 1336. For example, the first edge 1338 may be a distal edge while the second edge 1336 may be a proximal edge, or vice-versa. Electrode 1315 may be, for example, generally cylindrical (e.g., ring-like) in shape. In some embodiments, electrode 1315 may include a circular, ellipsoidal, flattened ellipse, or oval cross-section.

As described above, first edge 1338 can include a contoured pattern. For instance, edge 1338 may have at least one peak and at least one valley. For instance, a distance H_{P} between a peak point P_{P} located on edge 1338 and a closest point p₂ located on edge 1336 is larger than a corresponding distance H_{V} between a valley point P_{V} located on an edge 1338 and an associated closest point p₁ located on edge 1336. In an embodiment, points P_{P} and P_{V} can be connected by a smooth curve without any discontinuities, while in an alternative embodiment, points P_{P} and P_{V} can be connected with a curve including one or more discontinuities and/or one or both of points P_{P} and P_{V} can be a discontinuity point.

Edges 1336 and 1338, as shown in FIG. 14, are only illustrative and other contoured edges may be used. For example, edge 1336 may be similarly contoured as edge 1338. In some embodiments, contours of edges 1336 and 1338 may include multiple peaks and valleys. For example, a contour of edge 1338 may include a repeating pattern, such as, for example, a sinusoidal pattern. In some embodiments, the edges 1336 and 1338 may be symmetric or not symmetric relative to each other and/or different portions of edges 1336 and 1338 can be symmetric relative to other portions of edges 1336 and 1338.

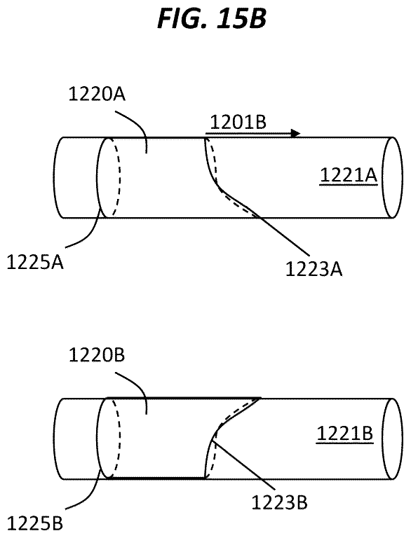

For illustrating the localized effect of contoured electrodes on the electric field, FIGS. 15A and 15B depict two arrangements of electrodes (e.g., on neighboring splines). FIG. 15A is a schematic view of electrodes 1210A, 1210B disposed on respective parallel splines 1211A, 1211B (e.g., parallel splines 1211A and 1211B may be approximations of splines of an ablation device 110 in a basket configuration). Electrodes 1210A, 1210B may be ring electrodes. First electrode 1210A may be mounted on first spline 1211A while second electrode 1210B may be mounted on second spline 1211B. In some embodiments, the splines 1211A and 1211B may be composed of a polymeric material. In demonstrating a computational model of an electric field produced by the electrodes, the electrodes may be surrounded by a blood pool and a voltage potential difference of about 1 kV may be applied across the electrodes 1210A, 1210B. In FIGS. 15A and 15B, diameters and lengths (along a longitudinal axes 1201A and 1201B) of electrodes 1210A, 1210B are respectively the same. As depicted, electrodes 1210A, 1210B do not include any contoured edges. In particular, electrode 1210A includes first and second edges 1213A, 1215A that are not contoured, and electrode 1210B includes first and second edges 1213B, 1215B that are not contoured.

FIG. 15B is a schematic view of electrodes 1220A, 1220B disposed on parallel splines 1221A, 1221B (e.g., parallel splines 1221A and 1221B may be approximations of splines of an ablation device 110 in a basket configuration). Electrode 1220A has an upper edge contour (first edge) 1223A, and electrode 1220B has an upper edge contour (first edge) 1223B. Each of the edges 1223A, 1223B can have a single valley and for exemplary purposes, a tangent discontinuity at a peak. In other embodiments, the edge contour can be continuous at the peak. Electrodes 1220A, 1220B also have lower edges 1225A, 1225B that are not contoured. In FIG. 15B, the diameters of the electrodes 1220A, 1220B and the length (along a longitudinal axis of each spline 1221A, 1221B) of the electrodes 1220A, 1220B are respectively the same for the two electrodes 1220A, 1220B. In demonstrating a computational model of an electric field produced by the electrodes, the electrodes 1220A, 1220B may be surrounded by a blood pool and a voltage potential difference of about 1 kV may be applied across the electrodes 1220A, 1220B.

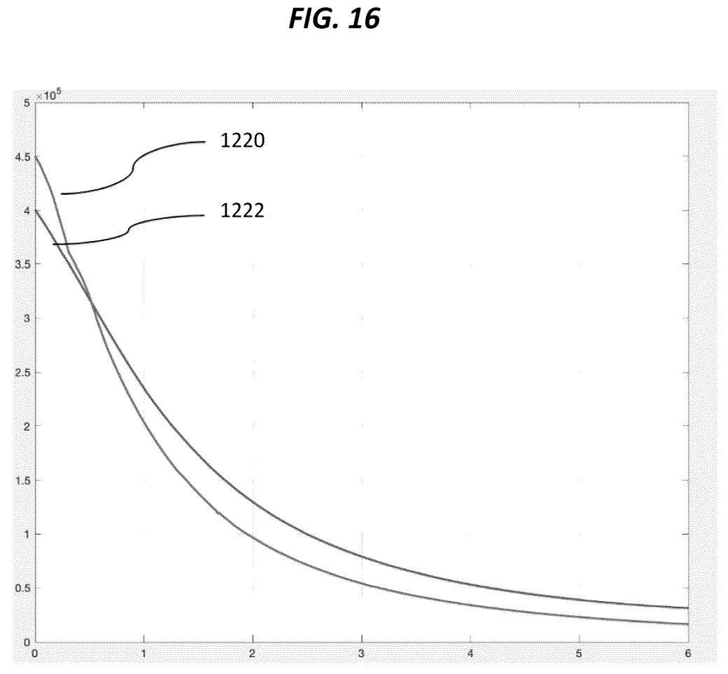

FIG. 16 is a graph showing electric field intensity along lines 1201A, 1201B in FIGS. 15A and 15B. Line 1201A can be an imaginary line that runs parallel to a longitudinal axis of spline 1211A, and line 1201B can be an imaginary line that runs parallel to a longitudinal axis of spline 1221A. Lines 1201A, 1201B can be equal and about 600 μm in length. As noted above, to demonstrate the electric field produced by the electrodes, it can be assumed that electrodes 1210A, 1210B, 1220A, 1220B are positioned in a conductive medium (e.g., a blood pool) and that a voltage potential difference of about 1 kV is applied across electrodes 1210A, 1210B as a pair and electrodes 1220A, 1220B as a pair. FIG. 16 shows the difference in the electric field intensity along lines 1201A, 1201B as a result of the differing electrode geometries. In FIG. 16, the horizontal axis starts at the interface between the electrode and the material of the spline (e.g., electrode-polymer interface) and extends longitudinally parallel to the polymer surface of each spline.

As shown in FIG. 16, plot line 1220 represents the electric field intensity of electrodes 1210A and 1210B with non-contoured edges 1213A and 1213B, while plot line 1222 represents the electric field intensity of electrodes 1221A and 1221B with contoured edges 1223A and 1223B. As shown in FIG. 16, the contoured electrodes have a smaller electric field intensity than the non-contoured electrodes near the electrode-spline interface (e.g., electrode-polymer interface).

Furthermore, the gradient or drop-off of the electric field intensity is less steep for the contoured electrodes than the non-contoured electrodes. This relationship generally applies for other contoured edge geometries as described herein relative to non-contoured electrode geometries. Accordingly, as depicted in FIG. 16, systems, devices, and methods described herein including electrodes with contoured edges can reduce the electric field intensity at the contoured edge of the electrodes and reduce the drop-off of the electric field intensity, both of which can improve the safe delivery of irreversible electroporation in medical applications (e.g., cardiac ablation procedures).

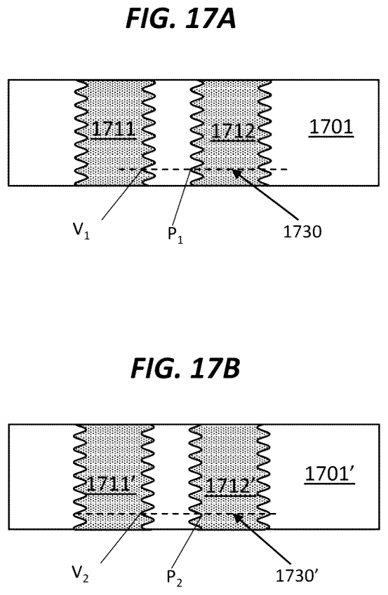

In some embodiments, multiple electrodes disposed on a shaft or spline can have electrodes each with contoured edges. FIGS. 17A and 17B show examples of ablation devices 1701 and 1701′ with respective electrodes 1711, 1712, and 1711′, 1712′, according to embodiments. In use, electrodes 1711, 1712 can be energized as an electrode pair, and similarly electrodes 1711′, 1712′ can be energized as an electrode pair, for delivering pulsed field ablation. Electrodes 1711, 1712, 1711′, and 1712′ may include contoured edges. As depicted in FIG. 17A, electrodes 1711, 1712 can have contoured edges that are synchronized, i.e., a valley (e.g., a valley V₁) of electrode 1711 can be synchronized with a peak (e.g., a peak P₁) of electrode 1712 along direction 1730. In an alternative arrangement, electrodes 1711′, 1712′ can have contoured edges that are non-synchronized, i.e., a valley (e.g., a valley V₂) of electrode 1711′ is synchronized with a peak (e.g., a peak P₂) of electrode 1712′ along direction 1730′, as shown in FIG. 17B.

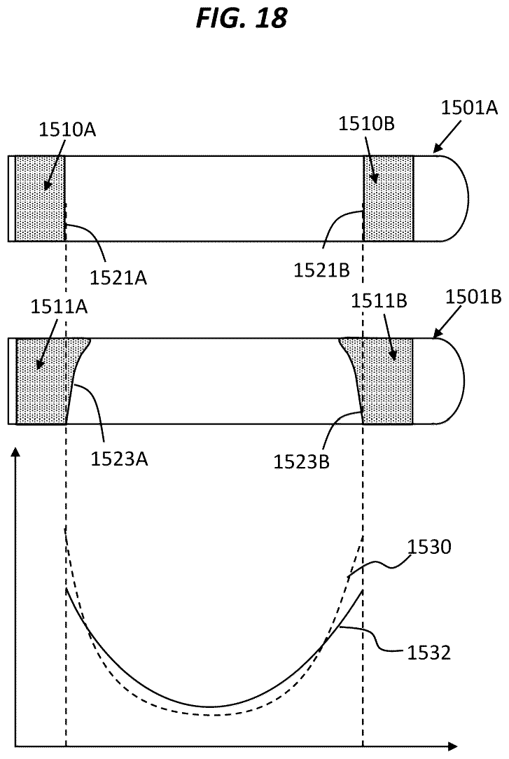

FIG. 18 schematically depicts adjacent electrodes with and without contoured edges, and plots of their respective electric field intensity between the adjacent electrodes. In particular, two adjacent electrodes 1510A and 1510B with respective non-contoured edges 1521A and 1521B are disposed on a shaft or spline 1501A, and two adjacent electrodes 1511A and 1511B with respective contoured edges 1523A and 1523B are disposed on a shaft or spline 1501B. Plot lines 1530 and 1532 illustratively depict the difference in the electric field intensity along the longitudinal axis of the shafts 1501A, 1501B in the space between the adjacent electrodes 1510A, 1510B, 1511A, 1511B. As shown, the contoured electrodes 1511A, 1511B can have a smaller electric field intensity than the non-contoured electrodes 1510A, 1510B near the electrode-polymer interface. Furthermore, the gradient or drop-off of the electric field intensity is less steep for the contoured electrodes 1511A, 1511B than the non-contoured electrodes 1510A, 1510B.

### Methods

Also, consistent with disclosed embodiments, a method for ablating tissue is also provided. In some embodiments, the method includes delivering an ablation device to a cardiac chamber of a heart of a patient, deploying the ablation device (e.g., positioning the device near a target site, deploying the device, etc.), and delivering a pulse waveform to the ablation device such that the ablation device generates a pulsed electric field for ablating tissue.

In various embodiments, system 100 may be used to ablate tissue using various methods described herein. In an example embodiment, the tissue ablation is performed in or near one or more heart chamber(s) using the systems and devices described herein. In an embodiment, the heart chamber(s) may be the left atrial chamber and include its associated pulmonary veins, while the devices and methods described herein can also be used in other cardiac chambers. Generally, one or more catheters may be advanced in a minimally invasive fashion through vasculature to a target location. For example, an ablation device may be advanced through vasculature over a guidewire and through a deflectable sheath. The sheath may be configured for deflection and aid in guiding a focal ablation catheter through vasculature and one or more predetermined targets (e.g., pulmonary vein ostia). A dilator may be advanced over a guidewire and configured for creating and dilating a transseptal opening during and/or prior to use. The methods described here include introducing and disposing an ablation device (e.g., ablation device) in contact with one or more pulmonary vein ostial or antral regions. Optionally, a pacing signal may be delivered to the heart using a cardiac stimulator (e.g., cardiac stimulator) and/or measure cardiac activity. Spatial characteristics (e.g., position, orientation, configuration) of the ablation device and tissue may be determined and used to generate an expected ablation zone and/or tissue map for display. A pulse waveform may be delivered by one or more electrodes of the ablation device to ablate tissue. The tissue map including ablated tissue and the expected ablation zone may be updated in real-time on the display as the device is navigated through tissue and additional pulse waveforms are delivered to the tissue.

In some embodiments, ablation energy may be delivered in synchrony with cardiac pacing. In some embodiments, the voltage pulse waveforms described herein may be applied during a refractory period of the cardiac cycle so as to avoid disruption of the sinus rhythm of the heart. Alternatively, the ablation energy can be delivered asynchronously.

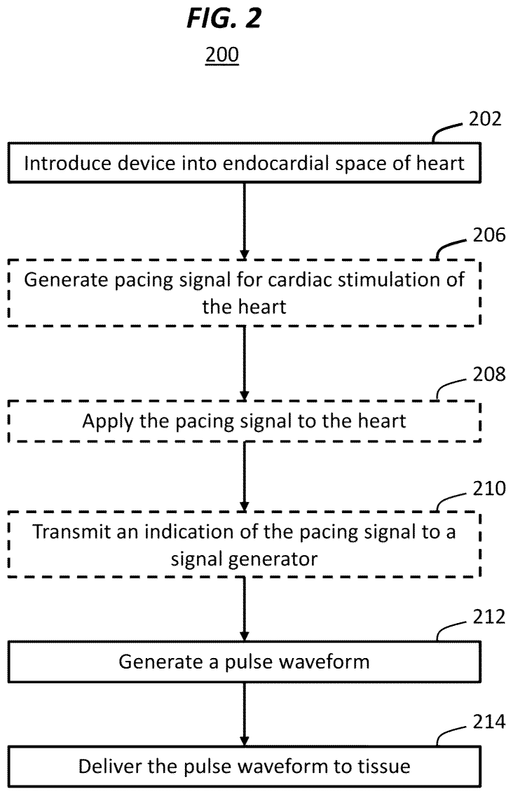

FIG. 2 is an example process 200 of a tissue ablation. Optional steps of the process 200 are indicated in FIG. 2 by dashed lines. The process 200 includes introduction of a device (e.g., ablation device) into an endocardial space of a heart at step 202. In some embodiments, the device may be advanced to be disposed in contact with a pulmonary vein ostium. For example, electrodes of an ablation device may form an approximately circular arrangement of electrodes disposed in contact with an inner radial surface at a pulmonary vein ostium. In some embodiments, the electrodes of the ablation device can be disposed near or adjacent to an endocardial surface of the heart. Optionally, a portion of the ablation device can be deployed (e.g., splines are deployed into a basket, or a balloon is expanded). Optionally, a pacing signal may be generated for cardiac stimulation of the heart at step 206. The pacing signal may then be applied to the heart at step 208. For example, the heart may be electrically paced with a cardiac stimulator to ensure pacing capture to establish periodicity and predictability of the cardiac cycle. One or more of atrial and ventricular pacing may optionally be applied. An indication of the pacing signal may be transmitted to a signal generator at step 210. A time window within the refractory period of the cardiac cycle may then be defined within which one or more voltage pulse waveforms may be delivered. In some embodiments, a refractory time window may follow a pacing signal. For example, a common refractory time window may lie between both atrial and ventricular refractory time windows.

A pulse waveform may be generated, e.g., in synchronization with the pacing signal at step 212 in the case when a pacing signal is employed. For example, a voltage pulse waveform may be applied in a refractory time window. In some embodiments, the pulse waveform may be generated with a time offset with respect to the indication of the pacing signal. For example, the start of a refractory time window may be offset from the pacing signal by a time offset. The voltage pulse waveform(s) may be applied over a series of heartbeats over corresponding common refractory time windows. In other embodiments, a pulse waveform may be generated and applied asynchronously. The generated pulse waveform may be delivered to tissue at step 214. In some embodiments, the pulse waveform may be delivered to pulmonary vein ostium of a heart of a patient via one or more splines of a set of splines of an ablation device. In other embodiments, voltage pulse waveforms as described herein may be selectively delivered to electrode subsets such as anode-cathode subsets for ablation and isolation of the pulmonary vein. For example, a first electrode of a group of electrodes may be configured as an anode and a second electrode of the group of electrodes may be configured as a cathode. These steps may be repeated for a desired number of pulmonary vein ostial or antral regions to have been ablated (e.g., 1, 2, 3, 4 or more ostia). In alternate embodiments, the ablation pulse waveform can be delivered without a pacing signal, i.e., asynchronously delivered.

It should be understood that the examples and illustrations in this disclosure serve exemplary purposes and departures and variations such as numbers of splines, number of electrodes, and so on, or a variety of focal ablation devices such as linear ablation catheters etc. can be built and deployed according to the teachings herein without departing from the scope of this invention.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Some embodiments described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Field Programmable Gate Arrays (FPGAs), Read-Only Memory (ROM) and Random-Access Memory (RAM) devices. Other embodiments described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

The specific examples and descriptions herein are exemplary in nature and embodiments may be developed by those skilled in the art based on the material taught herein without departing from the scope of the present invention.

1. A pulsed field ablation catheter comprising:
a first shaft defining a longitudinal axis and a lumen;
a second shaft disposed within the lumen and having a distal portion that extends from a distal end of the first shaft;
a set of splines coupled to the first shaft and the second shaft, the set of splines configured to transition from an undeployed configuration into an expanded configuration in which the set of splines bow radially outward from the longitudinal axis of the first shaft; and
a plurality of electrodes configured to generate an electric field for ablating tissue, the plurality of electrodes including one or more contoured electrodes, each contoured electrode configured as a generally cylindrical ring disposed about a respective spline of the set of splines and including a proximal edge and an opposite distal edge, wherein one or both of the proximal edge and the distal edge defines a plurality of peaks and valleys extending circumferentially about the respective spline.

2. The pulsed field ablation catheter of claim 1, wherein each contoured electrode is configured to reduce an electric field intensity at an interface between the contoured edge of that contoured electrode and the spline on which that contoured electrode is disposed.

3. The pulsed field ablation catheter of claim 2, wherein each contoured electrode is configured to reduce a drop-off in the electric field intensity in a direction extending away from the contoured edge of that contoured electrode along a longitudinal axis of the spline on which that contoured electrode is disposed.

4. The pulsed field ablation catheter of claim 1, wherein each contoured electrode is a most distal electrode on the spline on which that contoured electrode is disposed.

5. The apparatus of claim 4, wherein the distal edge of each contoured electrode is the contoured edge.

6. The pulsed field ablation catheter of claim 4, wherein the proximal edge of each contoured electrode is not a contoured edge and lies within a single plane.

7. The pulsed field ablation catheter of claim 1, wherein the proximal and distal edges each define a plurality of peaks and valleys extending circumferentially about the respective spline.

8. The pulsed field ablation catheter of claim 7, wherein each peak of the proximal edge is aligned with a peak of the distal edge.

9. The pulsed field ablation catheter of claim 7, wherein each peak of the proximal edge is aligned with a valley of the distal edge.

10. The pulsed field ablation catheter of claim 1, wherein the peaks and valleys of the contoured edge have a periodic pattern.

11. The pulsed field ablation catheter of claim 1, wherein each valley has a radius of curvature of about 10 μm to about 50,000 μm, and wherein each peak has a radius of curvature of less than about 500 μm.

12. The pulsed field ablation catheter of claim 1, wherein a radius of curvature of each peak differs from a radius of curvature of each valley.

13. A pulsed field ablation catheter comprising:
a linear shaft defining a longitudinal axis; and
a plurality of electrodes disposed on a distal portion of the linear shaft, the plurality of electrodes configured to generate an electric field for ablating tissue, the plurality of electrodes including:
a tip electrode disposed at a distal end of the linear shaft, the tip electrode including a first contoured edge; and
a set of proximal electrodes disposed proximal of the tip electrode, the set of proximal electrodes including a contoured electrode having a second contoured edge, the first and second contoured edges each defining a plurality of peaks and valleys extending circumferentially about the linear shaft.

14. The pulsed field ablation catheter of claim 13, wherein each of the first and second contoured edges is configured to reduce an electric field intensity at an interface between that contoured edge and the linear shaft.

15. The pulsed field ablation catheter of claim 14, wherein each of the first and second contoured edges is configured to reduce a drop-off in the electric field intensity in a direction extending away from that contoured edge along a longitudinal axis of the linear shaft.

16. The pulsed field ablation catheter of claim 13, wherein the first contoured edge is a proximal edge of the tip electrode, and the second contoured edge is a distal edge of the proximal electrode of the set of proximal electrodes that is nearest to the tip electrode.

17. The pulsed field ablation catheter of claim 16, wherein each peak of the first contoured edge is aligned with a peak of the second contoured edge.

18. The pulsed field ablation catheter of claim 16, wherein each peak of the first contoured edge is aligned with a valley of the second contoured edge.

19. The pulsed field ablation catheter of claim 13, wherein the distal tip electrode is configured to be energized with a first polarity and the plurality of proximal electrodes is configured to be energized with a second polarity opposite to the first polarity to form an electrode pair for generating the electric field.

20. The pulsed field ablation catheter of claim 13, further comprising a sleeve disposable over a portion of the linear shaft, the sleeve movable relative to the linear shaft to expose at least a subset of the set of proximal electrodes such that the subset of proximal electrodes can be used to generate the electric field.
